(19) United States
(12) Patent Application Publication
(10) Pub. No.: US 2025/0163379 A1
(43) Pub. Date: May 22, 2025

(54) METHOD FOR DIFFERENTIATING HEMATOPOIETIC STEM/PROGENITOR CELLS INTO NK CELLS

(71) Applicant: HEMACELL BIOTECHNOLOGY INC., Suzhou, Jiangsu (CN)

(72) Inventor: Chen WU, Suzhou Jiangsu (CN)

(21) Appl. No.: 18/840,854

(22) PCT Filed: Mar. 1, 2023
(86) PCT No.: PCT/CN2023/078940

§ 371 Date: Aug. 22, 2024

(30) Foreign Application Priority Data
Mar. 2, 2022 (CN) 202210203293.8

(51) Int. Cl. C12N 5/0783

(52) U.S. Cl. CPC C12N 5/0646; C12N 2501/125; C12N 2501/2303; C12N 2501/2307; C12N 2501/2315; C12N 2501/26; C12N 2501/845; C12N 2501/999; C12N 2506/11

(57) ABSTRACT

The present invention relates to a method for inducing proliferation and/or differentiation of hematopoietic stem/progenitor cells, the method comprising activating the y-aminobutyric acid pathway of hematopoietic stem/progenitor cells. The present invention also provides a culture medium for inducing proliferation and/or differentiation of hematopoietic stem/progenitor cells into NK cells and use thereof.

### TECHNICAL FIELD

The present application relates to the field of biomedicine, and specifically to a method for differentiating hematopoietic stem/progenitor cells into NK cells.

### BACKGROUND

NK cells are a type of lymphocytes that are different from T cells and B cells. They can exert cell-killing activity without being sensitized, which is not MHC-restricted and does not depend on antibodies, so it is called natural killer activity. There are a series of special activating and inhibiting receptors on the surface of NK cells, which can activate or inhibit their killing effect. NK cells are easily activated by factors secreted by tumors to exert cell-killing effects. They can destroy target tumor cells by secreting tumor necrosis factor and perforin. In addition, NK cells can be expanded in vitro, but due to the characteristics of NK cells themselves, it is difficult to genetically modify them (the efficiency of lentiviral vector infection is low). Therefore, it is of great significance to obtain NK cells through in vitro differentiation of stem cells.

Hematopoietic stem cell/progenitor cells (HSPCs) are adult stem and progenitor cells in the blood that have the ability to differentiate and proliferate into one or more blood cell lineages. To date, there is no reported method for differentiating NK cells from hematopoietic stem and progenitor cells in vitro.

### SUMMARY

The present application provides a method for differentiating hematopoietic stem/progenitor cells into NK cells. The differentiation method provided in the present application introduces the γ-aminobutyric acid (GABA) signaling pathway, which can enhance the differentiation ability of hematopoietic stem/progenitor cells and promote their differentiation into common lymphoid progenitors.

In one aspect, the present application provides a method for inducing proliferation and/or differentiation of hematopoietic stem/progenitor cells (HSPC), which comprises activating the γ-aminobutyric acid (GABA) signaling pathway of the hematopoietic stem/progenitor cells.

In another aspect, the present application provides a method for inducing proliferation and/or differentiation of hematopoietic stem/progenitor cells into common lymphoid progenitors (CLPs), which comprises activating the γ-aminobutyric acid (GABA) signaling pathway of the hematopoietic stem/progenitor cells.

In another aspect, the present application provides a method for inducing differentiation of hematopoietic stem/progenitor cells into natural killer (NK) cells, which comprises activating the GABAA and/or GABAC receptors of the hematopoietic stem/progenitor cells.

In some embodiments, the method comprises activating the GABAA and/or GABAC signaling pathway in the hematopoietic stem/progenitor cells.

In some embodiments, the method comprises administering a GABA pathway activator to the hematopoietic stem/progenitor cells.

In some embodiments, the present application provides a method for inducing proliferation and/or differentiation of hematopoietic stem/progenitor cells into NK cells, which comprises administering a GABA pathway activator to the hematopoietic stem/progenitor cells, wherein the concentration of the GABA pathway activator is about 0.1-1000 mM.

In some embodiments, the GABA pathway activator comprises a GABAA pathway activator and/or a GABAC pathway activator.

In some embodiments, the GABA pathway activator comprises a GABA receptor agonist.

In some embodiments, the GABA receptor agonist comprises a GABAA receptor agonist and/or a GABAC receptor agonist.

In some embodiments, the GABAA receptor agonist comprises one or more selected from the group consisting of Abecarnil, Barbiturates, Eszopiclone, Bamaluzole, Fengabine, γ-aminobutyric acid (GABA), trans-4-aminocrotonic acid (TACA), Gabamide, GABOB, Gaboxadol, Ibotenic acid, Isoguvacine, Isonipecotic acid, Muscimol, Phenibut, Picamilon, Progabide, Propofol, Quisqualamine, SL75102, Thiomuscimol, Topiramate and Zolpidem.

In some embodiments, the GABAA receptor agonist is 7-aminobutyric acid (GABA).

In some embodiments, the GABAA receptor agonist is trans-4-aminocrotonic acid (TACA).

In some embodiments, the GABAC receptor agonist comprises one or more selected from the group consisting of cis-4-aminocrotonic acid (CACA), (+)-cis-2-(aminomethyl)cyclopropanecarboxylic acid (CAMP), γ-aminobutyric acid (GABA), trans-4-aminocrotonic acid (TACA), γ-amino-β-hydroxybutyric acid (GABOB), N4-chloroacetylcytosine arabinoside, Picamilon, Progabide and Tolgabide.

In some embodiments, the GABAC receptor agonist is γ-aminobutyric acid (GABA).

In some embodiments, the GABAC receptor agonist is trans-4-aminocrotonic acid (TACA).

In some embodiments, the GABA pathway activator comprises a positive allosteric enhancer (PAM) of a GABA receptor.

In some embodiments, the positive allosteric enhancer of the GABA receptor comprises a positive allosteric enhancer of the GABAA receptor and/or a positive allosteric enhancer of the GABAC receptor.

In some embodiments, the positive allosteric enhancer of the GABAA receptor comprises one or more selected from the group consisting of Alcohols, Avermectins, Barbiturates, Benzodiazepines, Bromides, Carbamates, Chloral hydrate, chloralose, petrichloral and other 2,2,2-trichloroethanol prodrugs, Chlormezanone, Clomethiazole, Dihydroergolines, Etazepine, Etifoxine, 2-Substituted phenols, Imidazoles, Kavalactones, Loreclezole, Neuroactive steroids, Nonbenzodiazepines, Propofol, Piperidinediones, Propanidid, Pyrazolopyridines, Quinazolinones, Skullcap constituents, Stiripentol, Disulfonylalkanes, Valerian constituents and Volatile organic compounds.

In some embodiments, the GABA pathway activator comprises GABA and/or GABA derivatives.

In some embodiments, the GABA pathway activator comprises one or more selected from the group consisting of trans-4-aminocrotonic acid (TACA), muscol, (±)-trans-2-(aminomethyl)cyclopropanecarboxylic acid (TAMP), trans-2-methyl-4-aminocrotonic acid (2-MeTACA), 3-(aminomethyl)-1-oxo-1-hydroxyphosphane (3-AMOHP), 3-(amino)-1-oxo-1-hydroxyphosphane (3-AOHP), 3-(guanidino)-1-oxo-1-hydroxy-phosphate (3-GOHP), 4-aminocyclopent-1-enecarboxamide (4-ACPAM) and 4-amino-N-hydroxycyclopent-1-enecarboxamide (4-ACPHA).

In some embodiments, the GABA pathway activator comprises one or more selected from the group consisting of Muscimol, (R)-Baclofen, (RS)-Baclofen, SKF 97541, Acamprosate calcium, Thiomuscimol, Flurazepam, Flumazenil, 3-Aminopropylphosphonic Acid, AWD 131-138, Isoguvacine, TB 21007, Kojic Amine, Progabide, SL 75102, 3-APSA, MRK 016, TP 003, L-838,417, MK 0343, RuBi GABA trimethylphosphine, RuBi-GABA, Abecarnil, Barbiturate, Eszopiclone, Bamaluzole, Gabamide, Ibotenic acid, Isonipecotic acid, Phenibut, Picamilon, Propofol, Quisqualamine, Topiramate, Zolpidem, 1,4-Butanediol, γ-Butyrolactone, γ-Hydroxybutyric acid, γ-Hydroxyvaleric acid, γ-Valerolactone, Lesogaberan, Phenibut, 4-Fluorophenibut, Tolgabide, N4-Chloroacetylcytosine arabinoside, Methionine, Gabapentin, Piperazine, Gabapentin HCl, Etomidate, 6-Hydroxyflavone, 3,4,5-Trimethoxycinnamic acid (TMCA), Glabridin, Afloqualone and Oxiracetam.

In some embodiments, the GABA pathway activator further comprises one or more selected from the group consisting of 4-aminobutanoic acid (GABA), γ-Amino-β-hydroxybutyric acid (GABOB), trans-4-aminocrotonic acid (TACA), and muscimol, cis-4-amino-crotonic acid(CACA), (+)-cis-2-(aminomethyi)cyclopropane carboxylic acid (CAMP), (±)-trans-2-(aminomethyl) cyclopropane carboxylic acid (TAMP), trans-2-methyl-4-aminocrotonic acid (2-MeTACA), 3-(aminomethyl)-1-oxo-1-hydroxy-phospholane (3-AMOHP), 3-(amino)-1-oxo-1-hydroxy-phospholane (3-AOHP), 3-(guanidino)-1-oxo-1-hydroxy-phosphoiane (3-GOHP), 4-aminocyclopent-1-enecarboxamide (4-ACPAM), 4-amino-N-hydroxycyclopent-1-enecarboxamide (4-ACPHA).

In some embodiments, the concentration of the GABA pathway activator is about 0.1-1000 nM.

In some embodiments, the concentration of the GABA pathway activator is about 10-20 nM.

In some embodiments, the method comprises culturing the hematopoietic stem/progenitor cells using an initial differentiation medium, wherein the initial differentiation medium comprises an NK differentiation basal medium and a GABA pathway activator.

In some embodiments, the initial differentiation medium further comprises one or more of the following cytokines: SCF, IL3, IL7, IL15 and Flt3-L.

In some embodiments, the method further comprises using an NK differentiation medium, wherein the NK differentiation medium comprises an NK differentiation basal medium and one or more cytokines selected from the group consisting of SCF, IL3, IL7, IL15 and Flt3-L.

In some embodiments, the concentration of the cytokine is about 1-50 ng/mL.

In some embodiments, the NK differentiation basal medium comprises one or more selected from the group consisting of. DMEM high glucose medium, Ham's F-12Nutrient Mix, GlutaMAX™ supplement medium, heat-inactivated human AB serum, L-glutamic acid, 2-mercaptoethanol, sodium selenite, aminoethanol and L-ascorbic acid.

In some embodiments, the NK differentiation medium further comprises a GABA pathway activator.

In some embodiments, the NK differentiation medium comprises NK differentiation medium I, and the NK differentiation medium I comprises a basal differentiation medium and one or more cytokines selected from the group consisting of SCF, IL3, IL7, IL15 and Flt3-L.

In some embodiments, the NK differentiation medium comprises NK differentiation medium II, and the NK differentiation medium II comprises a basal differentiation medium and one or more cytokines selected from the group consisting of SCF, IL15, IL7 and Flt3-L.

In some embodiments, in the initial differentiation medium, the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL.

In some embodiments, in the initial differentiation medium, the concentration of SCF is about 15-25 ng/mL; the concentration of IL3 is about 3-7 ng/mL; the concentration of IL7 is about 15-25 ng/mL; the concentration of IL15 is about 8-12 ng/mL; and the concentration of Flt3-L is about 8-12 ng/mL.

In some embodiments, in the NK differentiation medium I, the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL.

In some embodiments, in the NK differentiation medium I, the concentration of SCF is about 15-25 ng/mL; the concentration of IL3 is about 3-7 ng/mL; the concentration of IL7 is about 15-25 ng/mL; the concentration of IL15 is about 8-12 ng/mL; and the concentration of Flt3-L is about 8-12 ng/mL.

In some embodiments, in the NK differentiation medium II, the concentration of SCF is about 1-50 ng/mL; the concentration of IL15 is about 1-50 ng/mL; the concentration of IL7 is about 1-50 ng/mL; and the concentration of Flt3-L is about 1-50 ng/mL.

In some embodiments, in the NK differentiation medium II, the concentration of SCF is about 15-25 ng/mL; the concentration of IL15 is about 8-12 ng/mL; the concentration of IL7 is about 15-25 ng/mL; and the concentration of Flt3-L is about 8-12 ng/mL.

In some embodiments, the method comprises the following steps: 1) inoculating the hematopoietic stem/progenitor cells in an initial differentiation medium for culture, wherein the initial differentiation medium comprises a GABA pathway activator; 2) after completing step 1), continuing the culture using NK differentiation medium I, wherein the NK differentiation medium I comprises cytokines SCF, IL3, IL7, IL15 and Flt3-L in a basal differentiation medium; and 3) continuing the culture using NK differentiation medium II, wherein the NK differentiation medium II comprises cytokines SCF, IL3, IL7, IL15 and Flt3-L in a basal differentiation medium.

In some embodiments, the method comprises the following steps: 1) inoculating hematopoietic stem and progenitor cells in an initial differentiation medium and culturing for about 5 days, wherein the initial differentiation medium is a culture medium obtained by adding cytokines SCF, IL3, IL7, IL15, Flt3-L and a GABA pathway activator to a basal differentiation medium; 2) continuing the culture for about 3-5 days using NK differentiation medium I, wherein the NK differentiation medium I is a culture medium obtained by adding cytokines SCF, IL3, IL7, IL15 and Flt3-L to a basal differentiation medium; 3) replacing the culture medium by half using NK differentiation medium II and continuing the culture; during the culture, the culture medium is replaced by half using the NK differentiation medium II every about 5-6 days, the system is cultured for about 22-28 days or until NK cells are obtained, wherein the NK differentiation medium II is a culture medium obtained by adding cytokines SCF, IL15, IL7 and Flt3-L to the basal differentiation medium.

In some embodiments, the NK differentiation medium I further comprises a GABA pathway activator.

In some embodiments, the NK differentiation medium II further comprises a GABA pathway activator.

In some embodiments, in step 1), the culture conditions are about 35-39° C. and about 3-7% CO₂.

In some embodiments, the method comprises increasing the expression, function and/or activity of a GABA receptor in the hematopoietic stem/progenitor cells.

In some embodiments, the GABA receptor comprises a GABAA receptor and/or a GABAC receptor.

In some embodiments, the method comprises increasing the expression, function and/or activity of a GABA receptor in the hematopoietic stem/progenitor cells through gene regulation.

In some embodiments, the gene regulation comprises one or more of the following ways: constructing an exogenous gene sequence for expression in cells, and gene editing to regulate the up-regulation and/or activation of endogenous genes.

In some embodiments, the hematopoietic stem/progenitor cells are derived from induced pluripotent stem cells.

In some embodiments, the hematopoietic stem/progenitor cells are derived from human blood in vitro.

In some embodiments, the hematopoietic stem/progenitor cells are derived from umbilical cord blood.

In some embodiments, the hematopoietic stem/progenitor cells are derived from bone marrow.

In some embodiments, the hematopoietic stem/progenitor cells are CD34+ hematopoietic stem/progenitor cells.

In another aspect, the present application further provides a culture medium comprising a GABA pathway activator.

In some embodiments, the GABA pathway activator comprises a GABA receptor agonist and/or a positive allosteric enhancer of a GABA receptor.

In some embodiments, the GABA pathway activator includes GABA and/or GABA derivatives.

In some embodiments, the culture medium further comprises a basal differentiation medium and one or more cytokines.

In some embodiments, the cytokines include SCF, IL3, IL7, IL15 and Flt3-L.

In some embodiments, the basal differentiation medium comprises DMEM high glucose medium, Ham's F-12Nutrient Mix, GlutaMAX™ supplement medium, heat-inactivated human AB serum, L-glutamic acid, 2-mercaptoethanol, sodium selenite, aminoethanol and L-ascorbic acid.

In another aspect, the present application further provides a medium kit comprising an initial differentiation medium and an NK differentiation medium, wherein the initial differentiation medium comprises a GABA pathway activator.

In some embodiments, in the medium kit, the initial differentiation medium further comprises a basal differentiation medium and cytokines SCF, IL3, IL7, IL15 and Flt3-L.

In some embodiments, in the medium kit, in the initial differentiation medium, the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL.

In some embodiments, in the medium kit, in the initial differentiation medium, the concentration of SCF is about 15-25 ng/mL; the concentration of IL3 is about 3-7 ng/mL; the concentration of IL7 is about 15-25 ng/mL; the concentration of IL15 is about 8-12 ng/mL; and the concentration of Flt3-L is about 8-12 ng/mL.

In some embodiments, in the medium kit, the NK differentiation medium comprises NK differentiation medium I and NK differentiation medium II.

In some embodiments, in the medium kit, the NK differentiation medium I comprises a basal differentiation medium and cytokines SCF, IL3, IL7, IL15 and Flt3-L.

In some embodiments, in the medium kit, in the NK differentiation medium I, the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL.

In some embodiments, in the medium kit, in the NK differentiation medium I, the concentration of SCF is about 15-25 ng/mL; the concentration of IL3 is about 3-7 ng/mL; the concentration of IL7 is about 15-25 ng/mL; the concentration of IL15 is about 8-12 ng/mL; and the concentration of Flt3-L is about 8-12 ng/mL.

In some embodiments, in the medium kit, the NK differentiation medium II comprises a basal differentiation medium and cytokines SCF, IL15, IL7 and Flt3-L.

In some embodiments, in the medium kit, in the NK differentiation medium II, the concentration of SCF is about 1-50 ng/mL; the concentration of IL15 is about 1-50 ng/mL; the concentration of IL7 is about 1-50 ng/mL; and the concentration of Flt3-L is about 1-50 ng/mL.

In some embodiments, in the medium kit, in the NK differentiation medium II, the concentration of SCF is about 15-25 ng/mL; the concentration of IL15 is about 8-12 ng/mL; the concentration of IL7 is about 15-25 ng/mL; and the concentration of Flt3-L is about 8-12 ng/mL.

In some embodiments, the NK differentiation medium of the medium kit further comprises a GABA pathway activator.

In another aspect, the present application further provides use of the culture medium and/or the medium kit in differentiating NK cells from hematopoietic stem/progenitor cells.

In another aspect, the present application further provides a composition comprising hematopoietic stem/progenitor cells and the culture medium or the medium kit.

In another aspect, the present application further provides a common lymphoid progenitor differentiated from hematopoietic stem/progenitor cells by activation of a GABA pathway.

In another aspect, the present application further provides an NK cell differentiated from hematopoietic stem/progenitor cells by activation of a GABA pathway.

In another aspect, the present application further provides a reagent for NK cell differentiation, which comprises a GABA pathway activator.

In another aspect, the present application further provides use of a GABA pathway activator in preparing a reagent for NK cell differentiation.

In another aspect, the present application further provides use of a GABA pathway activator in the differentiation and/or expansion of hematopoietic stem/progenitor cells into NK cells.

In another aspect, the present application further provides use of a GABA pathway activator in the production of a culture medium for differentiation of stem cells into NK cells.

In another aspect, the present application further provides a culture platform for obtaining NK cells derived from hematopoietic stem/progenitor cells, which comprises the method, culture medium, medium kit according to the present application, and/or hematopoietic stem/progenitor cells.

Those skilled in the art can easily discern other aspects and advantages of the present application from the detailed description below. Only exemplary embodiments of the present application are shown and described in the following detailed description. As those skilled in the art will realize, the contents of the present application enable those skilled in the art to make changes to the specific embodiments disclosed without departing from the spirit and scope of the invention covered by the present application. Accordingly, the drawings and descriptions in the specification of the present application are illustrative only and not restrictive.

### BRIEF DESCRIPTION OF DRAWINGS

The specific features of the invention to which the present application relates are set forth in the appended claims. The features and advantages of the invention to which the present application relates can be better understood by reference to the exemplary embodiments described in detail below and the drawings. A brief description of the drawings is as follows:

FIG. 1 shows that the GABA activator according to the present application promotes the production of more NK cells.

FIG. 2 shows that the GABA activator according to the present application induces the production of more CD16+CD56+ cytotoxic NK cells.

FIG. 3 shows that the GABA activator according to the present application induces the production of more CD314+ NK cells.

FIG. 4 shows that the GABA activator according to the present application induces the production of more CD335+ NK cells.

FIG. 5 shows that the GABA activator according to the present application induces the production of more CD336+ NK cells.

### DETAILED DESCRIPTION

The embodiments of the invention of the present application will be described below with specific examples. Those skilled in the art can easily understand other advantages and effects of the invention of the present application from the disclosure of the specification.

### Definition of Terms

In the present application, the term “derivative” generally means another chemical substance structurally related to a chemical substance, or a chemical substance that can be prepared by another chemical substance (i.e., a chemical substance that derives the chemical substance), for example by chemical or enzymatic modification. In the present application, the term “derivative” may refer to one with a structure which is derived from the parent compound and which is similar in structure to the parent compound. A derivative may exhibit similar functions and/or activities as the parent compound.

In the present application, the term “including” generally means the meaning of comprising, covering, containing, or encompassing. In some cases, it also means “being” and “consisting of”.

For the purposes of the present application, the terms “and/or” shall be understood to mean any one, two, or more of the alternatives, or any combination thereof.

In the present application, the term “about” generally refers to a change in a range of 0.5-10% greater or less than a specified value, for example, a change in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% greater or less than a specified value.

In the present application, the term “proliferation” generally means increasing the number of cells of a particular type or multiple types of cells from cells of a starting population, which may be different or the same. The starting cells used for proliferation need not be the same as the cells resulting from the proliferation. For example, the proliferated cells can result from the growth and differentiation of a starting population of cells.

In the present application, the term “differentiation” generally refers to the process by which non-specific or less specific cells acquire the characteristic of a particular cell. Differentiated or differentiation-induced cells are cells that have taken up a more specific position in a cell lineage.

In the present application, that term “marker phenotype” generally refers to the identification of a marker or antigen on a cell to determine their phenotype (e.g., differentiation status and/or cell type). For example, immunophenotyping can be used, which uses antibodies to recognize antigens presented on cells. Antibodies can be either monoclonal or polyclonal and those having minimal cross-reactivity with other cellular markers are usually selected. These markers that identify the same cell type among species can be identified based on the same markers, which may differ in structure (e.g., amino acid sequence) among species. Cell markers may include cell differentiation markers, and gene expression markers. Gene expression markers may include expressed genes that are indicative of cell type or differentiation state.

In the present application, the term “GABAA” receptor generally refers to a pentamer protein that forms a membrane ion channel. The “GABAA” receptor according to the present application also covers variants, homologues, analogs and/or derivatives thereof.

In the present application, the term “GABAC” receptor may also be referred to as GABA-p receptor, GABA-r, GABRR, typically a homologous pentamer ligand gated ion channel composed of p subunits. The “GABAC” receptors according to the present application also cover variants, homologues, analogs and/or derivatives thereof.

In the present application, the term “stem cell” generally refers to a self-replicating and pluripotent cell having one or more of the following properties: (1) long-term self-renewal, or the ability to produce at least one identical copy of a primordial cell, (2) differentiation into a plurality of cell types at the single-cell level and, in some cases, only one specialized cell type, and (3) functional regeneration of tissue in vivo. Stem cells are subdivided into totipotent, pluripotent, multipotent and oligo/unipotent according to their developmental potential.

In the present application, the term “progenitor cell” also generally has the ability to self-renew and differentiate into more mature cells, but is directed to a certain lineage (e.g., hematopoietic progenitor cells are directed to the blood lineage; medullary progenitor cells are directed to the bone marrow lineage; lymphoid progenitor cells are directed to the lymph lineage), whereas stem cells do not have to be so restricted.

Hematopoietic stem cells (HSCs) give rise to committed hematopoietic progenitor cells (HPCs) that contribute to the mature blood cell pool throughout the life of an organism. The term “hematopoietic stem cell” or “HSC” refers to multipotent stem cells that produce all blood cell types of an organism, including bone marrow lineages (e.g., monocytes and macrophages, neutrophils, basophils, eosinophils, red blood cells, megakaryocytes/platelets, dendritic cells), and lymphoid lineages (e.g., T-cells, B-cells, NK-cells), as well as other lineages known in the art.

In the present application, the term “induced pluripotent stem cell” may generally be abbreviated to iPS cells or iPSC, and generally refers to a class of pluripotent stem cells prepared by an artificial means from non-pluripotent cells. For example, the artificial means may be the introduction of reprogramming factors. For example, the non-pluripotent cells can be adult somatic cells or terminally differentiated cells, such as fibroblasts, hematopoietic cells, muscle cells, neurons, epidermal cells.

In the present application, the term “GABA pathway activator” generally refers to any substance, e.g., small molecule, nucleic acids, capable of activating or enhancing the intracellular γ-aminobutyric acid (GABA) signaling pathway. The “GABA signaling pathway” according to the present application may include any signal processor involved in the GABA-related signaling pathway, including its upstream signaling pathway and/or its downstream signaling pathway. Exemplary GABA pathway activators include GABA molecules and may also include GABA derivatives. For example, the GABA pathway activator may comprise a GABA receptor agonist. In the present application, the term “agonist” generally refers to a reagent that results in increased expression and/or activity of a target gene or protein. Agonists can bind to and activate their cognate receptors in some form, which can directly or indirectly lead to physiological effects on target genes or proteins.

In the present application, the term “cytokine” generally refers to a compound or component (e.g., an autoimmune factor) produced by a cell and affecting the physiological state of the cell (itself) or other cells that produce cytokines. Cytokines also include any compounds or components produced by recombinant or synthetic processing, which products have similar structure and/or biological activity to their naturally occurring forms. For example, the cytokines also encompass truncations, functionally active fragments, homologues, analogs, and variants thereof.

The term “composition” used herein generally refers to products comprising a specified amount of a specified ingredient, and any products produced directly or indirectly from a combination of specified amounts of specified ingredients. In the present application, the composition may further include other inactive ingredients, for example, carriers, excipients, adjuvants, and stabilizers. In the present application, the term “ex vivo” generally refers to the manipulation of cells, tissues and/or organs that have been removed from the body of the organism. In some embodiments, the cells, tissues and/or organs can be returned to the organism by certain methods, or introduced into another organism.

In the present application, the term “in vitro” generally refers to the removal or release of a portion of an organism from the organism.

In the present application, the term “in vivo” usually refers to being inside an organism. In some case, for example, that term “in vivo” may refer to a particular location in the tissue or organ under test.

### DETAILED DESCRIPTION OF THE INVENTION

### Methods

In an aspect, the present application provides a method for inducing proliferation and/or differentiation of hematopoietic stem/progenitor cells, which comprises activating the γ-aminobutyric acid (GABA) signaling pathway of the hematopoietic stem/progenitor cells.

In another aspect, the present application provides a method for inducing proliferation and/or differentiation of stem cells, which comprises activating the γ-aminobutyric acid (GABA) signaling pathway of the stem cells.

In another aspect, the present application provides a method for inducing proliferation and/or differentiation of hematopoietic stem/progenitor cells into common lymphoid progenitors (CLPs), which comprises activating the γ-aminobutyric acid (GABA) signaling pathway of the hematopoietic stem/progenitor cells.

In another aspect, the present application provides a method for inducing proliferation and/or differentiation of hematopoietic stem/progenitor cells into natural killer cells (NK cells), which comprises activating the γ-aminobutyric acid (GABA) signaling pathway of the hematopoietic stem/progenitor cells.

In another aspect, the present application provides a method for inducing proliferation and/or differentiation of hematopoietic stem/progenitor cells into NK cells, which comprises administering a GABA pathway activator to the hematopoietic stem/progenitor cells. For example, the concentration of the GABA pathway activator is about 0.1-1000 mM. For example, the concentration of the GABA pathway activator is about 0.1-900 mM, about 0.1-900 mM, about 0.1-800 mM, about 0.1-700 mM, about 0.1-600 mM, about 0.1-500 mM, about 0.1-400 mM, about 0.1-300 mM, about 0.1-200 mM, about 0.1-100 mM, about 1-50 mM, about 5-20 mM, about 10-100 mM, about 20-200 mM, about 20-300 mM, about 30-400 mM, about 30-500 mM, about 30-600 mM, about 50-700 mM, about 100-800 mM, about 200-800 mM, about 300-900 mM, about 400-900 mM, about 500-900 mM, about 600-1000 mM, about 700-1000 mM, about 800-900 mM or about 900-1000 mM.

In the present application, the GABA signaling pathway in the method may comprise a GABAA and/or a GABAC signaling pathway.

For example, the cells are hematopoietic stem cells. For example, the cells are hematopoietic progenitor cells. For example, the cells are stem cells. For example, the cells are hematopoietic stem/progenitor cells. For example, the GABA signaling pathway is a GABAA signaling pathway. For example, the GABA signaling pathway is a GABAC signaling pathway.

In the present application, the activation of the GABA pathway may be achieved by regulating the expression, function and/or activity of upstream and downstream related proteins of the GABA pathway.

In the present application, the method of inducing proliferation and/or differentiation of hematopoietic stem/progenitor cells into natural killer cells (NK cells) may comprise increasing the expression, function and/or activity of GABA receptors in the hematopoietic stem/progenitor cells.

For example, the expression level of GABA receptors in the hematopoietic stem/progenitor cells is up-regulated by about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 20%, about 30%, about 40%, about 50%, about 60%, about 70%, about 80%, about 90%, about 100%, about 150%, about 200%, about 300% compared to unmodified hematopoietic stem/progenitor cells.

For example, the activity of GABA receptors in the hematopoietic stem/progenitor cells is up-regulated by about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 20%, about 30%, about 40%, about 50%, about 60%, about 70%, about 80%, about 90%, about 100%, about 150%, about 200%, about 300% compared to unmodified hematopoietic stem/progenitor cells.

For example, the function of GABA receptors in the hematopoietic stem/progenitor cells is up-regulated by about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 20%, about 30%, about 40%, about 50%, about 60%, about 70%, about 80%, about 90%, about 100%, about 150%, about 200%, about 300% compared to unmodified hematopoietic stem/progenitor cells.

In the present application, the GABA signaling pathway activated in hematopoietic stem/progenitor cells may be activated at the gene level, at the transcription level, at the translation level, and/or at the protein level.

For example, it can be activated by applying chemical regulatory means or by genetic regulatory means.

For example, the chemical modulation means may include administering a GABA pathway activator. The GABA pathway activator may directly or indirectly activate the GABA pathway. For example, the GABA pathway activator may act directly on the GABA receptor. For example, the GABA pathway activator may indirectly cause activation of the GABA pathway by acting on other pathways in the cell.

For example, the gene regulation method may include using CRISPR to regulate endogenous gene activation and/or up-regulated expression. For example, the CRISPR/Cas9 system can be used to regulate endogenous gene activation and/or up-regulated expression.

For example, it can be achieved by introducing an exogenous protein or an exogenous nucleic acid molecule encoding the protein, or by causing increased expression of an endogenous protein or an endogenous gene encoding the protein. For example, the up-regulated expression of the GABA receptor may be caused by a mutation in the regulatory region of the gene encoding the receptor. In certain instances, the up-regulated expression of the GABA receptor may be achieved by altering the function of one or more components of the translation and/or transcription processes.

For example, the gene regulation means can be through small activating RNA (saRNA) means.

In the present application, the method may include a process of differentiating hematopoietic stem/progenitor cells into common lymphoid progenitors, and/or a process of differentiating common lymphoid progenitors into NK cells.

In the present application, the method may include a process of differentiating stem cells into common lymphoid progenitors, and/or a process of differentiating common lymphoid progenitors into NK cells.

In the present application, the method may comprise the following steps: 1) inoculating the hematopoietic stem/progenitor cells in an initial differentiation medium for culture, wherein the initial differentiation medium comprises a GABA pathway activator; 2) after completing step 1), continuing the culture using NK differentiation medium I, wherein the NK differentiation medium I comprises cytokines SCF, IL3, IL7, IL15 and/or Flt3-L in a basal differentiation medium; and 3) continuing the culture using NK differentiation medium II, wherein the NK differentiation medium II comprises cytokines SCF, IL3, IL7, IL15 and/or Flt3-L in a basal differentiation medium.

In the present application, the method may comprise the following steps: 1) inoculating hematopoietic stem and progenitor cells in an initial differentiation medium and culturing for about 5 days, wherein the initial differentiation medium is a culture medium obtained by adding cytokines SCF, IL3, IL7, IL15, Flt3-L and a GABA pathway activator to a basal differentiation medium; 2) continuing the culture for about 3-5 days using NK differentiation medium I, wherein the NK differentiation medium I is a culture medium obtained by adding cytokines SCF, IL3, IL7, IL15 and Flt3-L to a basal differentiation medium; 3) replacing the culture medium by half using NK differentiation medium II and continuing the culture; during the culture, the culture medium is replaced by half using the NK differentiation medium II every about 5-6 days, the system is cultured for about 22-28 days or until NK cells are obtained, wherein the NK differentiation medium II is a culture medium obtained by adding cytokines SCF, IL15, IL7 and Flt3-L to the basal differentiation medium.

For example, in step 1), the culture condition may be about 35-39° C. For example, the culture conditions are about 35° C., 35.5° C., 36° C., 36.5° C., 37° C., 37.5° C., 38° C., 38.5° C. or 39° C.

For example, in step 1), the culture conditions may be culture at about 3-7% CO₂, for example, the culture conditions may be culture at about 3%, 3.5%, 4%, 4.5%, 5%, 5.5%, 6%, 6.5%, or 7% CO₂.

In the present application, the NK differentiation medium may also contain a GABA pathway activator.

In some embodiments, the method may be an in vitro method.

In some embodiments, the method may be an in vivo method.

In some embodiments, the method may be an ex vivo method.

In the present application, the method may be one not aimed at the diagnosis and treatment of diseases.

### GABA Pathway Activators

In the present application, the GABA pathway activator may include substances that can activate the GABA pathway. For example, the substance may be a chemical compound, a nucleic acid molecule and/or a protein. For example, the substance may be a macromolecular substance or a small molecular substance. For example, the substance may be an organic compound or an inorganic compound.

In the present application, the GABA pathway activator may include a GABAA pathway activator.

In the present application, the GABA pathway activator may include a GABAC pathway activator.

In the present application, the GABA pathway activator may include a GABA receptor agonist. For example, the GABA receptor agonist may include a GABAA receptor agonist. For example, the GABA receptor agonist may include a GABAC receptor agonist.

In the present application, the GABA receptor agonist may include GABA or derivatives thereof. In the present application, the GABA receptor agonist may include non-GABA or derivatives thereof, and such compounds may also activate the GABA pathway. For example, the GABAA receptor agonist may comprise one or more selected from the group consisting of Abecarnil, Barbiturates, Eszopiclone, Bamaluzole, Fengabine, γ-aminobutyric acid (GABA), trans-4-aminocrotonic acid (TACA), Gabamide, GABOB, Gaboxadol, Ibotenic acid, Isoguvacine, Isonipecotic acid, Muscimol, Phenibut, Picamilon, Progabide, Propofol, Quisqualamine, SL75102, Thiomuscimol, Topiramate and Zolpidem.

For example, the GABAA receptor agonist is γ-aminobutyric acid (GABA). For example, the GABAA receptor agonist is trans-4-aminocrotonic acid (TACA).

For example, the GABAC receptor agonist may comprise one or more selected from the group consisting of cis-4-aminocrotonic acid (CACA), trans-4-aminocrotonic acid (TACA), (+)-cis-2-(aminomethyl)cyclopropanecarboxylic acid (CAMP), γ-aminobutyric acid (GABA), γ-amino-o-hydroxybutyric acid (GABOB), N4-chloroacetylcytosine arabinoside, Picamilon, Progabide and Tolgabide.

For example, the GABAC receptor agonist is γ-aminobutyric acid (GABA). For example, the GABAC receptor agonist is trans-4-aminocrotonic acid (TACA).

In the present application, the GABA pathway activator may comprise a positive allosteric enhancer (PAM) of a GABA receptor. For example, the positive allosteric enhancer of the GABA receptor may comprise phenol, ketone, imidazole and/or pyrazole compounds.

For example, the positive allosteric enhancer of the GABA receptor may comprise a positive allosteric enhancer of the GABAA receptor. For example, the positive allosteric enhancer of the GABA receptor may comprise a positive allosteric enhancer of the GABAC receptor. For example, the positive allosteric enhancer of the GABAA receptor may comprise one or more selected from the group consisting of Alcohols, Avermectins, Barbiturates, Benzodiazepines, Bromides, Carbamates, Chloral hydrate, chloralose, petrichloral and other 2,2,2-trichloroethanol prodrugs, Chlormezanone, Clomethiazole, Dihydroergolines, Etazepine, Etifoxine, 2-Substituted phenols, Imidazoles, Kavalactones, Loreclezole, Neuroactive steroids, Nonbenzodiazepines, Propofol, Piperidinediones, Propanidid, Pyrazolopyridines, Quinazolinones, Skullcap constituents, Stiripentol, Disulfonylalkanes, Valerian constituents and Volatile organic compounds.

In the present application, the derivatives of the GABA pathway activator may comprise one or more selected from the group consisting of trans-4-aminocrotonic acid (TACA), muscol, (±)-trans-2-(aminomethyl)cyclopropanecarboxylic acid (TAMP), trans-2-methyl-4-aminocrotonic acid (2-MeTACA), 3-(aminomethyl)-1-oxo-1-hydroxyphosphane (3-AMOHP), 3-(amino)-1-oxo-1-hydroxyphosphane (3-AOHP), 3-(guanidino)-1-oxo-1-hydroxy-phosphate (3-GOHP), 4-aminocyclopent-1-enecarboxamide (4-ACPAM) and 4-amino-N-hydroxycyclopent-1-enecarboxamide (4-ACPHA).

In the present application, the GABA pathway activator may comprise one or more selected from the group consisting of Muscimol, (R)-Baclofen, (RS)-Baclofen, SKF 97541, Acamprosate calcium, Thiomuscimol, Flurazepam, Flumazenil, 3-Aminopropylphosphonic Acid, AWD 131-138, Isoguvacine, TB 21007, Kojic Amine, Progabide, SL 75102, 3-APSA, MRK 016, TP 003, L-838,417, MK 0343, RuBi GABA trimethylphosphine, RuBi-GABA, Abecarnil, Barbiturate, Eszopiclone, Bamaluzole, Gabamide, Ibotenic acid, Isonipecotic acid, Phenibut, Picamilon, Propofol, Quisqualamine, Topiramate, Zolpidem, 1,4-Butanediol, γ-Butyrolactone, γ-Hydroxybutyric acid, γ-Hydroxyvaleric acid, γ-Valerolactone, Lesogaberan, Phenibut, 4-Fluorophenibut, Tolgabide, N4-Chloroacetylcytosine arabinoside, Methionine, Gabapentin, Piperazine, Gabapentin HCl, Etomidate, 6-Hydroxyflavone, 3,4,5-Trimethoxycinnamic acid (TMCA), Glabridin, Afloqualone and Oxiracetam.

In the present application, the concentration of the GABA pathway activator may be about 0.1-1000 mM. For example, the concentration of the GABA pathway activator is about 0.1-900 mM, about 0.1-900 mM, about 0.1-800 mM, about 0.1-700 mM, about 0.1-600 mM, about 0.1-500 mM, about 0.1-400 mM, about 0.1-300 mM, about 0.1-200 mM, about 0.1-100 mM, about 1-50 mM, about 5-20 mM, about 10-20 mM, about 10-100 mM, about 20-200 mM, about 20-300 mM, about 30-400 mM, about 30-500 mM, about 30-600 mM, about 50-700 mM, about 100-800 mM, about 200-800 mM, about 300-900 mM, about 400-900 mM, about 500-900 mM, about 600-1000 mM, about 700-1000 mM, about 800-900 mM or about 900-1000 mM.

Culture medium/medium kits In the present application, the method may comprise culturing the hematopoietic stem/progenitor cells using an initial differentiation medium.

In the present application, the method may comprise using an NK differentiation medium.

In another aspect, the present application further provides a culture medium, and the culture medium may comprise a GABA pathway activator.

For example, the culture medium may comprise an initial differentiation medium. For example, the culture medium may contain one or more cytokines. For example, the cytokine may be selected from SCF, IL3, IL7, IL15 and Flt3-L.

For example, the culture medium may comprise an NK differentiation medium. For example, the culture medium may contain one or more cytokines. For example, the cytokine may be selected from SCF, IL3, IL7, IL15 and Flt3-L.

In another aspect, the present application further provides a medium kit, which may comprise an initial differentiation medium and an NK differentiation medium, wherein the initial differentiation medium comprises a GABA pathway activator.

In another aspect, the present application further provides a medium kit, which may comprise an initial differentiation medium and an NK differentiation medium, wherein the NK differentiation medium comprises a GABA pathway activator.

In another aspect, the present application further provides a medium kit, which may comprise an initial differentiation medium and an NK differentiation medium, wherein the initial differentiation medium comprises a GABA pathway activator, and the NK differentiation medium comprises a GABA pathway activator.

For example, the initial differentiation medium may include an NK differentiation basal medium and a GABA pathway activator. For example, the initial differentiation medium further comprises one or more of the following cytokines: SCF, IL3, IL7, IL15 and Flt3-L. For example, the initial differentiation medium may comprise SCF, IL3, IL7, IL15 and Flt3-L. For example, the concentration of the cytokine is about 1-50 ng/mL. For example, the concentration of the cytokine is about 1-50 ng/mL, about 1-45 ng/mL, about 1-40 ng/mL, about 1-30 ng/mL, about 3-7 ng/mL, about 8-12 ng/mL or about 15-25 ng/mL.

For example, in the initial differentiation medium, the concentration of SCF is about 15-25 ng/mL. For example, the concentration of SCF is about 15-25 ng/mL, about 15-24 ng/mL, about 15-23 ng/mL, about 15-22 ng/mL, about 15-21 ng/mL, about 15-22 ng/mL, about 16-25 ng/mL, about 17-25 ng/mL, about 18-25 ng/mL, about 19-25 ng/mL, about 20-25 ng/mL, about 21-25 ng/mL, about 22-25 ng/mL, about 23-25 ng/mL or about 24-25 ng/mL.

For example, in the initial differentiation medium, the concentration of IL3 is about 3-7 ng/mL. For example, the concentration of IL3 is about 3-6.5 ng/mL, about 3-6 ng/mL, about 3-5.5 ng/mL, about 3-5 ng/mL, about 3-4.5 ng/mL, about 3-4 ng/mL, about 3.5-7 ng/mL, about 4.5-7 ng/mL, about 5.5-7 ng/mL or about 6-7 ng/mL.

For example, in the initial differentiation medium, the concentration of IL7 is about 15-25 ng/mL. For example, the concentration of IL7 is about 15-25 ng/mL, about 15-24 ng/mL, about 15-23 ng/mL, about 15-22 ng/mL, about 15-21 ng/mL, about 15-22 ng/mL, about 16-25 ng/mL, about 17-25 ng/mL, about 18-25 ng/mL, about 19-25 ng/mL, about 20-25 ng/mL, about 21-25 ng/mL, about 22-25 ng/mL, about 23-25 ng/mL or about 24-25 ng/mL.

For example, in the initial differentiation medium, the concentration of IL15 is about 8-12 ng/mL. For example, the concentration of IL15 is about 8-11.5 ng/mL, about 8-11 ng/mL, about 8-10.5 ng/mL, about 8-10 ng/mL, about 8-9.5 ng/mL, about 8-9 ng/mL, about 8.5-12 ng/mL, about 9-12 ng/mL, about 9.5-12 ng/mL, about 10-12 ng/mL, about 10.5-12 ng/mL, about 11-12 ng/mL or about 11.5-12 ng/mL.

For example, in the initial differentiation medium, the concentration of Flt3-L is about 8-12 ng/mL. For example, the concentration of Flt3-L is about 8-11.5 ng/mL, about 8-11 ng/mL, about 8-10.5 ng/mL, about 8-10 ng/mL, about 8-9.5 ng/mL, about 8-9 ng/mL, about 8.5-12 ng/mL, about 9-12 ng/mL, about 9.5-12 ng/mL, about 10-12 ng/mL, about 10.5-12 ng/mL, about 11-12 ng/mL or about 11.5-12 ng/mL.

For example, the NK differentiation medium comprises an NK differentiation basal medium and one or more cytokines selected from the group consisting of SCF, IL3, IL7, IL15 and Flt3-L.

For example, the cytokine comprises SCF, IL3, IL7, IL15 and Flt3-L. For example, in the NK differentiation medium, the concentration of the cytokine is 1-50 ng/mL. For example, the concentration of the cytokine is about 1-50 ng/mL, about 1-45 ng/mL, about 1-40 ng/mL, about 1-30 ng/mL, about 3-7 ng/mL, about 8-12 ng/mL or about 15-25 ng/mL.

In the present application, the NK differentiation medium may include NK differentiation medium I, and the NK differentiation medium I may include a basal differentiation medium and one or more cytokines selected from the group consisting of SCF, IL3, IL7, IL15 and Flt3-L. For example, the NK differentiation medium I may further comprise a GABA pathway activator.

In the present application, the NK differentiation medium may include NK differentiation medium II, and the NK differentiation medium II may include a basal differentiation medium and one or more cytokines selected from the group consisting of SCF, IL15, IL7 and Flt3-L. For example, the NK differentiation medium II may further comprise a GABA pathway activator.

For example, in the NK differentiation medium I or in the NK differentiation medium II, the concentration of SCF is about 15-25 ng/mL. For example, the concentration of SCF is about 15-25 ng/mL, about 15-24 ng/mL, about 15-23 ng/mL, about 15-22 ng/mL, about 15-21 ng/mL, about 15-22 ng/mL, about 16-25 ng/mL, about 17-25 ng/mL, about 18-25 ng/mL, about 19-25 ng/mL, about 20-25 ng/mL, about 21-25 ng/mL, about 22-25 ng/mL, about 23-25 ng/mL or about 24-25 ng/mL.

For example, in the NK differentiation medium I or in the NK differentiation medium II, the concentration of IL3 is about 3-7 ng/mL. For example, the concentration of IL3 is about 3-6.5 ng/mL, about 3-6 ng/mL, about 3-5.5 ng/mL, about 3-5 ng/mL, about 3-4.5 ng/mL, about 3-4 ng/mL, about 3.5-7 ng/mL, about 4.5-7 ng/mL, about 5.5-7 ng/mL or about 6-7 ng/mL.

For example, in the NK differentiation medium I or in the NK differentiation medium II, the concentration of IL7 is about 15-25 ng/mL. For example, the concentration of IL7 is about 15-25 ng/mL, about 15-24 ng/mL, about 15-23 ng/mL, about 15-22 ng/mL, about 15-21 ng/mL, about 15-22 ng/mL, about 16-25 ng/mL, about 17-25 ng/mL, about 18-25 ng/mL, about 19-25 ng/mL, about 20-25 ng/mL, about 21-25 ng/mL, about 22-25 ng/mL, about 23-25 ng/mL or about 24-25 ng/mL.

For example, in the NK differentiation medium I or in the NK differentiation medium II, the concentration of IL15 is about 8-12 ng/mL. For example, the concentration of IL15 is about 8-11.5 ng/mL, about 8-11 ng/mL, about 8-10.5 ng/mL, about 8-10 ng/mL, about 8-9.5 ng/mL, about 8-9 ng/mL, about 8.5-12 ng/mL, about 9-12 ng/mL, about 9.5-12 ng/mL, about 10-12 ng/mL, about 10.5-12 ng/mL, about 11-12 ng/mL or about 11.5-12 ng/mL.

For example, in the NK differentiation medium I or in the NK differentiation medium II, the concentration of Flt3-L is about 8-12 ng/mL. For example, the concentration of Flt3-L is about 8-11.5 ng/mL, about 8-11 ng/mL, about 8-10.5 ng/mL, about 8-10 ng/mL, about 8-9.5 ng/mL, about 8-9 ng/mL, about 8.5-12 ng/mL, about 9-12 ng/mL, about 9.5-12 ng/mL, about 10-12 ng/mL, about 10.5-12 ng/mL, about 11-12 ng/mL or about 11.5-12 ng/mL.

For example, the NK differentiation basal medium may comprise one or more selected from the group consisting of: DMEM high glucose medium, Ham's F-12Nutrient Mix, GlutaMAX™ supplement medium, heat-inactivated human AB serum, L-glutamic acid, 2-mercaptoethanol, sodium selenite, aminoethanol and L-ascorbic acid.

### Cells

In the present application, the hematopoietic stem/progenitor cells may be derived from induced pluripotent stem cells.

In the present application, the hematopoietic stem/progenitor cells are derived from human blood ex vivo.

In the present application, the hematopoietic stem/progenitor cells are derived from umbilical cord blood.

In the present application, the hematopoietic stem/progenitor cells are derived from bone marrow.

In the present application, the hematopoietic stem/progenitor cells are CD34+ hematopoietic stem/progenitor cells.

In the present application, the differentiation pathway of the hematopoietic stem/progenitor cells may include differentiation from hematopoietic stem/progenitor cells to common lymphoid progenitors, and then differentiation from common lymphoid progenitors to NK cells.

In another aspect, the present application further provides an NK cell, which is differentiated from hematopoietic stem/progenitor cells by activation of a GABA pathway.

In another aspect, the present application further provides a common lymphoid progenitor differentiated from hematopoietic stem/progenitor cells by activation of a GABA pathway.

In the present application, the cells and/or the state of the cells can be determined by cell markers. For example, the type of cells and/or the state of cells can be determined by marker phenotype.

In some embodiments, the cells according to the present application are isolated.

In the present application, the cells can be pharmaceutically formulated according to any conventional method. For example, the active ingredient may be mixed or diluted with a carrier, an excipient or a diluent. Examples of suitable carriers, excipients or diluents are lactose, dextrose, sucrose, sorbitol, mannitol, glycine, polyethylene glycol, starch, gum Arabic, alginic acid, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. The preparation may additionally comprise, for example, fillers, anticoagulants, lubricants, wetting agents, flavoring agents, emulsifiers, preservatives, and the like. The compositions of the invention may be formulated by using any of the procedures known in the art so as to provide quick, sustained or delayed release of the active ingredient after administration to the patient.

The cells of the present application can be administered by injection (e.g. intramuscularly, intravenously, intraperitoneally, subcutaneously), or by other methods such as infusion to ensure that they enter the bloodstream in an effective form. The cells may also be administered via intratumoral, peritumoral, intralesional or perilesional routes to exert local and systemic therapeutic effects. For example, it may be administered topically or intravenously.

In the present application, the administration dose of the cells may also be a single dose or multiple doses. For example, the actual amount of cells administered may be determined based on a variety of relevant factors, such as the type of disease; the route of administration; the age, sex and/or weight of the patient; and the severity of the patient's symptoms.

### Composition, Reagent, Use

In another aspect, the present application further provides a composition comprising hematopoietic stem/progenitor cells and the culture medium or the medium kit.

In another aspect, the present application further provides a reagent for NK cell differentiation, which comprises a GABA pathway activator.

In another aspect, the present application further provides use of a GABA pathway activator in preparing a reagent for NK cell differentiation. In another aspect, the present application further provides use of a GABA pathway activator in the differentiation and/or expansion of hematopoietic stem/progenitor cells into NK cells.

In another aspect, the present application further provides use of a GABA pathway activator in the production of a culture medium for differentiation of stem cells into NK cells.

In another aspect, the present application further provides a culture platform for obtaining NK cells derived from hematopoietic stem/progenitor cells, which comprises the method, culture medium, medium kit according to the present application, and/or hematopoietic stem/progenitor cells.

Without wishing to be bound by any theory, the following examples are only for illustrating the methods, culture media and uses of the present application, and are not used to limit the scope of the invention of the present application.

### EXAMPLES

Experimental materials

| Name | Supplier | Cat. No. |
|---|---|---|
| GABA | Selleckchem | S4700 |
| TACA | Tocris | 0181 |
| SCF | PeproTech | 300-07 |
| IL3 | PeproTech | 200-03 |
| IL7 | PeproTech | 200-07 |
| IL15 | PeproTech | 200-15 |
| F1T3-L | PeproTech | 300-19 |
| DMEM high glucose | Gibco | C11995500BT |
| medium | | |
| DMEM/F12 medium | Gibco | 11330 |
| DMEM, High Glucose, | Gibco | 10566 |
| GlutaMAX ™ Supplement | | |
| L-glutamic Acid | Gibco | 35050 |
| Heat-inactivated | Sigma-Aldrich | H4522 |
| human AB serum | | |
| 2-mercaptoethanol | Gibco | 21985023 |
| Sodium selenite | Sigma-Aldrich | 214485 |
| Aminoethanol | Sigma-Aldrich | 398136 |
| L-ascorbic acid | Sigma-Aldrich | A5960 |
| CD56-APC antibody | BioLegend | 304610 |
| CD3-PE antibody | BioLegend | 300308 |
| CD16-PE antibody | BioLegend | 302008 |
| CD314-PE antibody | BioLegend | 320806 |
| CD335-PE antibody | BioLegend | 331907 |
| CD336-PE antibody | BioLegend | 325108 |
| DAPI | BioLegend | 422801 |

### Example 1 Inducing Differentiation of Hematopoietic Stem/Progenitor Cells into NK Cells

The experiment was carried out according to the following steps:
(1) Inoculating hematopoietic stem/progenitor cells into an initial differentiation medium and culturing at 35-39° C. and 3-7%₀CO₂ for 5 days;
(2) Continuing culturing cells in NIK differentiation medium I for 3-5 days;
(3) Continuing culturing the cells in NK differentiation medium II; replacing half of the culture medium every 5 to 6 days for 22 to 28 days or until NK cells are obtained.
(4) Basal differentiation medium: DMEM high glucose medium, Ham's F-12Nutrient Mix, GlutaMAX™ supplement medium, heat-inactivated human AB serum, L-glutamic acid, 2-mercaptoethanol, sodium selenite, aminoethanol and L-ascorbic acid.
(5) Initial differentiation medium: basal differentiation medium, SCF (15-25 ng/mL); IL3 (3-7 ng/mL); IL7 (15-25 ng/mL); IL15 (8-12 ng/mL); Flt3-L (8-12 ng/mL); GABA pathway activator.
(6) NK differentiation medium I: basal differentiation medium; SCF (15-25 ng/mL); IL3 (3-7 ng/mL); IL7 (15-25 ng/mL); IL15 (8-12 ng/mL); Flt3-L (8-12 ng/mL).
(7) NK differentiation medium II: basal differentiation medium; SCF (15-25 ng/mL); IL15 (8-12 ng/mL); IL7 (15-25 ng/mL); Flt3-L (8-12 ng/mL).

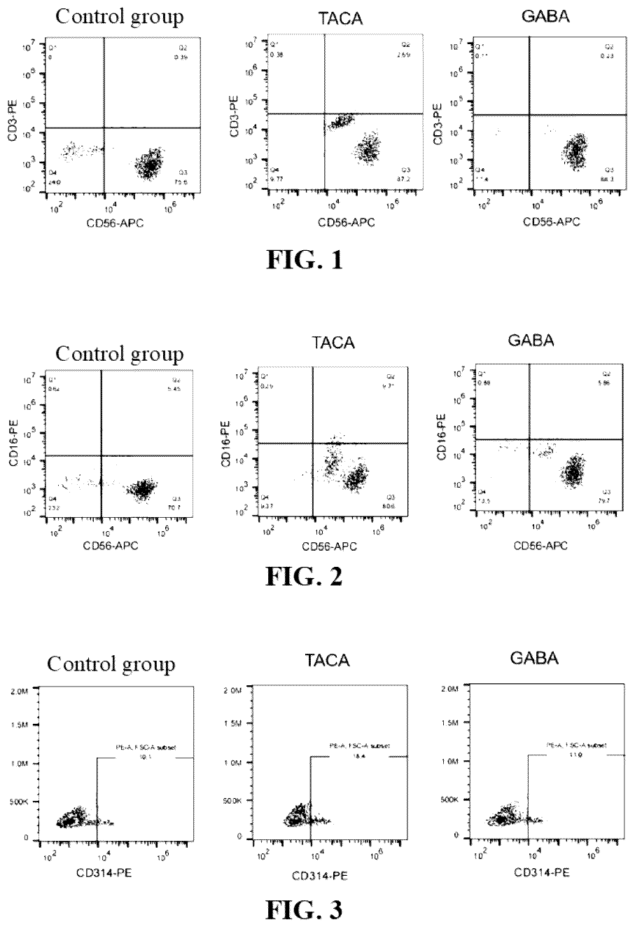

The experimental results are shown in FIG. 1:

CD3 is a marker of T cells, CD56 is a marker of NK cells, and the characterization of NK cells is CD3-CD56+. The efficiency of obtaining NK cells was 75.6%, 87.2%, and 88.3% for the control group, TACA and GABA, respectively.

The above results show that:

After adding GABA pathway activators to the culture medium, hematopoietic stem/progenitor cells can be induced to differentiate into common lymphoid progenitors more efficiently, and NK cells can be obtained more efficiently.

### Example 2 GABA Activator Promotes NK Cell Differentiation

Flow cytometry analysis of induced NK cells

(1) Collection of NK cells: The cells were centrifuged at 300 g for 5 min; the supernatant was discarded, an equal volume of FACS buffer was added to wash and resuspend the cells, and the cells were centrifuged; the above steps were repeated twice; the cells were centrifuged and the supernatant was discarded, and thereafter, 125 μL of FACS buffer was added to resuspend the cells.

(2) Cell staining: 2.5 μL of labeled antibody was added to the cell suspension (volume ratio of labeled antibody:cell=1:50), the system was mixed well, and allowed to react at room temperature in the dark for 30 min; after centrifugation, the supernatant was discarded; 1 mL of FACS buffer was added to wash and resuspend the cells, the cells were centrifuged, and the above steps were repeated twice; the cells were centrifuged and the supernatant was discarded, and thereafter 250 μL of FACS buffer was added to resuspend the cells, the system was transferred to a Falcon flow tube, and placed on ice; 0.25 μL of DAPI was added before loading, the system was mixed well for later loading and detection.

(3) Preparation of single-color compensation control: The microspheres were mixed evenly by inverting vigorously at least 10 times or pulse vortexing; to each EP tube, 1 drop of UltraCompe Beads was added, then 1 μL of fluorescent antibody conjugate was added, the system was well mixed by blowing-suction with a pipette, and incubated at room temperature in the dark for 30 min; 1 mL of FACS buffer was added to each EP tube, the system was centrifuged at 400 g for 5 min at room temperature; the supernatant was sucked out carefully without removing the beads, and then 250 μL of FACS buffer was added to each test tube; the sample was mixed evenly by flicking or pulse vortexing before analysis.

For most antibodies, amounts of 0.03-1.0 g provide suitable compensation values.

(4) Detection on the flow cytometer: Before detection, the stained cells were placed on ice to prevent cell aggregation; at least 10,000 cells were tested for each sample; unstained cells were analyzed on the flow cytometer, and appropriate FSC and side scatter SSC as well as the voltage of the fluorescence detector PMT were set.

(5) Analysis of the microsphere samples: the FSC/SSC were adjusted so that the microspheres were on the display interface; the FSC/SSC size can be adjusted to observe the microspheres; each single-color microsphere sample was analyzed to ensure that the positive peak was within the normal range; if the positive peak of the microsphere was beyond the normal range, the PMT voltage should be reduced as much as possible; all single-color microspheres were checked to ensure that the positive peak was within the normal range, and then the data was recorded; each single-color microsphere sample was analyzed, compensation settings were made, and the compensation control file was saved; the FSC/SSC of the cell sample were reset and the sample results were obtained; the sample results were collected and saved.

The results are shown in FIG. 2.

CD56 is a NK cell marker, CD16 is a cytotoxic marker of NK cells, and is one of the most characteristic activated NK cell receptors involved in anti-cancer immune response. A higher CD16+ level indicates a stronger NK cell function. The efficiency of obtaining CD16+CD56+ NK cells was 5.45%, 9.71%, and 5.86% for the control group, TACA and GABA, respectively.

The results are shown in FIG. 3.

CD314 is a cytotoxic marker of NK cells and one of the most characteristic activated NK cell receptors involved in anti-cancer immune response. A higher CD314+ level indicates a stronger NK cell function. The efficiency of obtaining CD314+ NK cells was 10.1%, 18.4%, and 11.0% for the control group, TACA and GABA respectively.

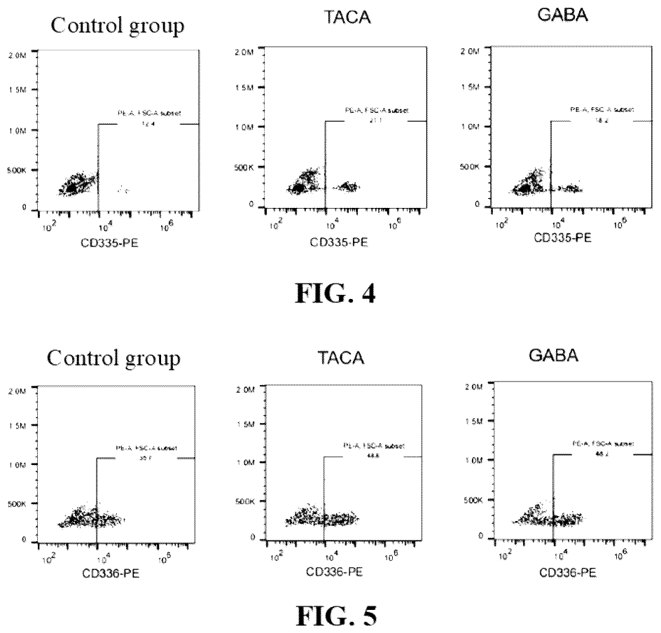

The results are shown in FIG. 4.

CD335 is a cytotoxic marker of NK cells and one of the main activation receptors of NK cells. A higher CD335+ level indicates a stronger NK cell function. The efficiency of obtaining CD335+ NK cells was 12.4%, 21.1%, and 18.2% for the control group, TACA and GABA respectively.

The results are shown in FIG. 5.

CD336 is a cytotoxic marker of NK cells and one of the main activation receptors of NK cells.

A higher CD336+ level indicates a stronger NK cell function. The efficiency of obtaining CD336+ NK cells was 35.7%, 48.8%, and 48.2% for the control group, TACA and GABA respectively.

The above results show that:

Compared with the control group, in the two groups with GABA activator induction (TACA and GABA), not only more NK cells were obtained, but also the obtained NK cells had stronger expression of cytotoxic markers, indicating that GABA activators promote NK cell differentiation.

1.-5. (canceled)

6. A method for inducing proliferation and/or differentiation of hematopoietic stem/progenitor cells into NK cells, comprising administering a GABA pathway activator to the hematopoietic stem/progenitor cells, wherein the concentration of the GABA pathway activator is about 0.1-1000 mM.

7. The method of claim 6, wherein the GABA pathway activator comprises a GABAA pathway activator and/or a GABAC pathway activator.

8. The method of claim 6, wherein the GABA pathway activator comprises a GABA receptor agonist, and/or a positive allosteric enhancer (PAM) of the GABA receptor.

9. The method according to claim 8, wherein the GABA receptor agonist comprises a GABAA receptor agonist, a GABAC receptor agonist, and wherein the positive allosteric enhancer of the GABA receptor comprises a positive enhancer of the GABAA receptor and/or a positive allosteric enhancer of the GABAC receptor.

10. The method according to claim 9, wherein the GABAA receptor agonist comprises one or more selected from the group consisting of Abecarnil, Barbiturates, Eszopiclone, Bamaluzole, Fengabine, γ-aminobutyric acid (GABA), Gabamide, γ-amino-β-hydroxybutyric acid (GABOB), Gaboxadol, Ibotenic acid, Isoguvacine, Isonipecotic acid, Muscimol, Phenibut, Picamilon, Progabide, Propofol, Quisqualamine, SL75102, Thiomuscimol, Topiramate, trans-4-aminocrotonic acid (TACA) and Zolpidem, wherein the GABAC receptor agonist comprises one or more selected from the group consisting of cis-4-aminocrotonic acid (CACA), (+)-cis-2-(aminomethyl)cyclopropanecarboxylic acid (CAMP), γ-aminobutyric acid (GABA), γ-amino-β-hydroxybutyric acid (GABOB), trans-4-aminocrotonic acid (TACA), N4-chloroacetylcvtosine arabinoside, Picamilon, Progabide and Tolgabide, and
wherein the positive allosteric enhancer of the GABAA receptor comprises one or more selected from the group consisting of Alcohols, Avermectins, Barbiturates, Benzodiazepines, Bromides, Carbamates, Chloral hydrate, chloralose, petrichloral and other 2,2,2-trichloroethanol prodrugs, Chlormezanone, Clomethiazole, Dihydroergolines, Etazepine, Etifoxine, 2-Substituted phenols, Imidazoles, Kavalactones, Loreclezole, Neuroactive steroids, Nonbenzodiazepines, Propofol, Piperidinediones, Propanidid, Pyrazolopyridines, Quinazolinones, Skullcap constituents, Stiripentol, Disulfonylalkanes, Valerian constituents and Volatile organic compounds.

11. The method of claim 9, wherein the GABAA receptor agonist is trans-4-aminocrotonic acid (TACA), or γ-aminobutyric acid (GABA), and wherein the GABAC receptor agonist is trans-4-aminocrotonic acid (TACA) or γ-aminobutyric acid (GABA).

12.-18. (canceled)

19. The method of claim 6, wherein the GABA pathway activator comprises GABA and/or GABA derivatives.

20. The method according of claim 6, wherein the GABA pathway activator comprises one or more selected from the group consisting of trans-4-aminocrotonic acid (TACA), muscol, (±)-trans-2-(aminomethyl)cyclopropanecarboxylic acid (TAMP), trans-2-methyl-4-aminocrotonic acid (2-MeTACA), 3-(aminomethyl)-1-oxo-1-hydroxyphosphane (3-AMOHP), 3-(amino)-1-oxo-1-hydroxyphosphane (3-AOHP), 3-(guanidino)-1-oxo-1-hydroxy-phosphate (3-GOHP), 4-aminocyclopent-1-enecarboxamide (4-ACPAM) and 4-amino-N-hydroxycyclopent-1-enecarboxamide (4-ACPHA).

21. The method according to claim 20, wherein the GABA pathway activator is trans-4-aminocrotonic acid (TACA).

22. The method of claim 6, wherein the GABA pathway activator comprises one or more selected from the group consisting of Muscimol, (R)-Baclofen, (RS)-Baclofen, SKF 97541, Acamprosate calcium, Thiomuscimol, Flurazepam, Flumazenil, 3-Aminopropylphosphonic Acid, AWD 131-138, Isoguvacine, TB 21007, Kojic Amine, Progabide, SL 75102, 3-APSA, MRK 016, TP 003, L-838,417, MK 0343, RuBi GABA trimethylphosphine, RuBi-GABA, Abecarnil, Barbiturate, Eszopiclone, Bamaluzole, Gabamide, Ibotenic acid, Isonipecotic acid, Phenibut, Picamilon, Propofol, Quisqualamine, Topiramate, Zolpidem, 1,4-Butanediol, γ-Butyrolactone, γ-Hydroxybutyric acid, γ-Hydroxyvaleric acid, γ-Valerolactone, Lesogaberan, Phenibut, 4-Fluorophenibut, Tolgabide, N4-Chloroacetylcytosine arabinoside, Methionine, Gabapentin, Piperazine, Gabapentin HCl, Etomidate, 6-Hydroxyflavone, 3,4,5-Trimethoxycinnamic acid (TMCA), Glabridin, Afloqualone and Oxiracetam.

23. (canceled)

24. The method of claim 6, wherein the concentration of the GABA pathway activator is about 10-20 nM.

25. The method of claim 6, comprising culturing the hematopoietic stem/progenitor cells using an initial differentiation medium, wherein the initial differentiation medium comprises an NK differentiation basal medium and a GABA pathway activator, wherein the initial differentiation medium further comprises one or more of the following cytokines: SCF, IL3, IL7, IL15 and Flt3-L, wherein the NK differentiation medium comprises an NK differentiation basal medium and one or more cytokines selected from the group consisting of SCF, IL3, IL7, IL15 and Flt3-L.

26.-30. (canceled)

31. The method of claim 25, wherein the NK differentiation medium comprises NK differentiation medium I, and the NK differentiation medium I comprises a basal differentiation medium and one or more cytokines selected from the group consisting of SCF, IL3, IL7, IL15 and Flt3-L.

32. The method of claim 25, wherein the NK differentiation medium comprises NK differentiation medium II, and the NK differentiation medium II comprises a basal differentiation medium and one or more cytokines selected from the group consisting of SCF, IL15, IL7 and Flt3-L.

33. The method of claim 25, wherein in the initial differentiation medium, the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL,
wherein in the NK differentiation medium I, the concentration of SCF is about 1-50 ng/mL; the concentration of IL3 is about 1-50 ng/mL: the concentration of IL7 is about 1-50 ng/mL: the concentration of IL15 is about 1-50 ng/mL; and the concentration of Flt3-L is about 1-50 ng/mL, and
wherein in the NK differentiation medium II, the concentration of SCF is about 1-50 ng/mL; the concentration of IL15 is about 1-50 ng/mL: the concentration of IL7 is about 1-50 ng/mL; and the concentration of Flt3-L is about 1-50 ng/mL.

34.-38. (canceled)

39. The method of claim 6, comprising the following steps: 1) inoculating the hematopoietic stem/progenitor cells in an initial differentiation medium for culture, wherein the initial differentiation medium comprises a GABA pathway activator; 2) after completing step 1), continuing the culture using NK differentiation medium I, wherein the NK differentiation medium I comprises cytokines SCF, IL3, IL7, IL15 and Flt3-L in a basal differentiation medium; and 3) continuing the culture using NK differentiation medium II, wherein the NK differentiation medium II comprises cytokines SCF, IL3, IL7, IL15 and Flt3-L in a basal differentiation medium.

40.-58. (canceled)

59. A medium kit comprising an initial differentiation medium and an NK differentiation medium, wherein the initial differentiation medium comprises a GABA pathway activator.

60. The medium kit according to claim 59, wherein the initial differentiation medium further comprises a basal differentiation medium and cytokines SCF, IL3, IL7, IL15 and Flt3-L.

61.-62. (canceled)

63. The medium kit of claim 59, wherein the NK differentiation medium comprises NK differentiation medium I and NK differentiation medium II, wherein the NK differentiation medium I comprises a basal differentiation medium and cytokines SCF, IL3, IL7, IL15 and Flt3-L, and wherein the NK differentiation medium II comprises a basal differentiation medium and cytokines SCF, IL15, IL7 and Flt3-L.

64. (canceled)

65. The medium kit of claim 63, wherein in the initial differentiation medium, NK differentiation medium I, and NK differentiation medium II the concentration of SCF is about 1-50 ng/mL; the concentration of IL3 is about 1-50 ng/mL; the concentration of IL7 is about 1-50 ng/mL; the concentration of IL15 is about 1-50 ng/mL; and the concentration of Flt3-L is about 1-50 ng/mL.

66.-79. (canceled)
